(19) United States
(12) Patent Application Publication
(10) Pub. No.: US 2010/0285593 A1
(43) Pub. Date: Nov. 11, 2010

(54) INTERNAL STANDARDS AND METHODS FOR USE IN QUANTITATIVELY MEASURING ANALYTES IN A SAMPLE

(75) Inventor: Mohamed Amoura, Luynes (FR)

Correspondence Address: EDWARDS ANGELL PALMER & DODGE LLP, P.O. BOX 55874, BOSTON, MA, 02205, US

(73) Assignee: WATERS TECHNOLOGIES CORPORATION, Milford, MA (US)

(21) Appl. No.: 12/676,011

(22) PCT Filed: Nov. 20, 2008
(86) PCT No.: PCT/US08/12938

§ 371 Date: Aug. 2, 2010

Related U.S. Application Data
(60) Provisional application No. 61/004,288, filed on Nov. 26, 2007.

(51) Int. Cl. G01N 31/00; G01N 30/02; G01N 33/00; G01N 21/00; G01N 1/00

(52) U.S. Cl. 436/8; 422/70; 436/86; 436/94; 436/164; 436/174

(57) ABSTRACT

The invention provides methods for quantitatively analyzing a plurality of analytes in a sample. Also described are general and specific internal standards useful in such analysis. In particular embodiments, these standards are described as useful in liquid chromatography/mass spectroscopy systems, which are also described herein. Moreover, in certain embodiments, the quantification methods of the present invention are useful in increasing the precision and/or accuracy of multiple analyte quantification for analytes contained in a single sample mixture using known analyte derivatives simultaneously analyzed, and compared to the unknown analytes.

### RELATED APPLICATION

This application claims priority to U.S. provisional application Ser. No. 61/004,288, filed Nov. 26, 2007, the entire disclosure of which is incorporated herein by this reference.

### BACKGROUND OF THE INVENTION

Chemical and biological samples often contain mixtures of a plurality of compounds. Such compounds may be independently or simultaneously characterized by a variety of analytical techniques, often combined with chromatographic techniques that may also be used to separate these mixtures for enhanced analysis.

Among the best-known chromatographic techniques are gas chromatography (GC), high performance liquid chromatography (HPLC) and super-critical fluid chromatography (SFC), with the most convenient being HPLC. In fact, HPLC methods are often used to separate a wide variety of polar and non-polar compounds; as the solvent (or mobile phase) and stationary phase that can be used in HPLC may be selected from a wide array of possibilities based upon the flexibility of the technique and the columns useful therein. In fact, with careful selection of the mobile phase and stationary phase, most sample mixtures can be separated into well-resolved peaks or fractions, which can subsequently be subjected to further analysis.

Characterization of these fractions by methods such as mass spectrometry (MS), e.g., LC/MS, provides qualitative information about the analytes present in the sample. In this way, it is often possible to identify the specific molecular species generating an MS signal by discerning its molecular weight (noting that different chemicals typically have different molecular weights). As such, MS is considered to be a very well utilized tool in the simultaneous qualitative characterization of one or more compounds in a single sample.

Yet, while MS is a powerful tool in the qualitative analysis of unknown organic compounds, there still remains a need for accurate and precise techniques that would allow the simultaneous quantification of a plurality of analytes contained in a single sample mixture.

### SUMMARY OF THE INVENTION

The present invention is directed to methods for quantitatively analyzing a plurality of analytes in a sample, internal standards for use in such analysis, and chromatography systems including these internal standards. Moreover, in certain embodiments, the quantification methods of the present invention are useful in increasing the precision and/or accuracy of multiple analyte quantification for analytes contained in a single sample mixture.

Accordingly, one aspect of the invention provides a method for quantitatively analyzing a plurality of analytes in a sample. The method comprises derivatizing analytes in a sample with a first derivatizing agent, e.g., AccQTag™, to form analyte derivatives in the sample, adding a known concentration of a plurality of analyte derivative standards to the sample to form a sample mixture, subjecting the sample mixture to chromatographic separation, detecting each individual analyte derivative and analyte derivative standard, and determining the quantity of each analyte derivative in the sample. The amount of each analyte derivative is determined by a response factor calculation in order to quantitatively analyze the plurality of analytes in the sample. In certain embodiments, the number of analytes in the sample is greater than 5, e.g., greater than 10, e.g., greater than 15, e.g., greater than 20, e.g., greater than 25, e.g., greater than 30, e.g., greater than 35, e.g., greater than 45.

In another aspect, the invention provides a method for quantitatively analyzing a plurality of amino acids in a sample. The method comprises derivatizing analytes in a sample with a first derivatizing agent to form amino acid derivatives in the sample, adding a known concentration of a plurality of amino acid derivative standards to the sample to form a sample mixture, subjecting the sample mixture to chromatographic separation, detecting each individual amino acid derivative and amino acid derivative standard, and determining the quantity of each amino acid derivative in the sample. The amount of each amino acid derivative is determined by a response factor calculation in order to quantitatively analyze the plurality of amino acids in the sample.

In an additional aspect, the invention provides a liquid chromatography/mass spectroscopy system for quantitatively analyzing the amount of a plurality of analytes in a sample. The system contains a chromatographic analysis system comprising a chromatographic column and a pump for pumping at least one mobile phase through the chromatographic column. The system also contains a mass spectroscopy analysis system comprising a mass spectrometer capable of detecting analyte derivatives, a first derivatizing agent useful for derivatizing analytes in a sample to form analyte derivatives in the sample comprising AccQTag™ or a functional derivative thereof, and a plurality of analyte derivative standards comprising AccQTag™ or a functional derivative thereof that have been labeled with a radioactive or stable isotope. In certain embodiments, the number of analyte derivative standards is greater than 10, e.g., greater than 15, e.g., greater than 20.

Another aspect of the invention provides a liquid chromatography/mass spectroscopy system for quantitatively analyzing the amount of a plurality of analytes in a sample. The system contains a chromatographic analysis system comprising a chromatographic column and a pump for pumping at least one mobile phase through the chromatographic column. The system also contains a mass spectroscopy analysis system comprising a mass spectrometer capable of detecting analyte derivatives, a first derivatizing agent useful for derivatizing analytes in a sample to form analyte derivatives in the sample comprising AccQTag™ or a functional derivative thereof, and reagents capable of producing a plurality of analyte derivative standards comprising AccQTag™ or a functional derivative thereof that have been labeled with a radioactive or stable isotope. In certain embodiments, the number of analyte derivative standards is greater than 10, e.g., greater than 15, e.g., greater than 20.

In yet another aspect, the invention provides a method of increasing precision of analyte quantification of a plurality of analytes. The method comprises derivatizing analytes in a sample with a first derivatizing agent to form analyte derivatives in the sample, adding a known concentration of a plurality of analyte derivative standards to the sample to form a sample mixture, subjecting the sample mixture to chromatographic separation, detecting each individual analyte derivative and analyte derivative standard, and determining the quantity of each analyte derivative in the sample. The amount of each analyte derivative is determined by a response factor calculation. As such, this method is useful for increasing the precision of analyte quantification of a plurality of analytes in the sample with respect to known methods.

Another aspect of the invention provides a method of increasing accuracy of analyte quantification of a plurality of analytes. The method comprises derivatizing analytes in a sample with a first derivatizing agent to form analyte derivatives in the sample, adding a known concentration of a plurality of analyte derivative standards to the sample to form a sample mixture, subjecting the sample mixture to chromatographic separation, detecting each individual analyte derivative and analyte derivative standard, and determining the quantity of each analyte derivative in the sample. The amount of each analyte derivative is determined by a response factor calculation. As such, this method is useful for increasing the accuracy of analyte quantification of a plurality of analytes in the sample with respect to known methods.

An additional aspect of the invention provides an internal standard reagent useful for quantitatively analyzing an analyte comprising AccQTag™ or a functional derivative thereof, which has been labeled with a radioactive or stable isotope.

In yet another aspect, the invention provides an internal standard useful for quantitatively analyzing an analyte prepared by reacting a known concentration of one or more analytes with AccQTag™ or a functional derivative thereof, which has been labeled with a radioactive or stable isotope.

### DETAILED DESCRIPTION OF THE INVENTION

The present invention is directed to methods for quantitatively analyzing a plurality of analytes in a sample. Also described are general and specific internal standards useful in such analysis. In particular embodiments, these standards are useful in liquid chromatography/mass spectroscopy systems, which are further described herein. Moreover, in certain embodiments, the quantification methods of the present invention are useful in increasing the precision and/or accuracy of quantification for multiple analytes contained in a single sample mixture using known analyte derivatives simultaneously analyzed, and compared to the derivatives prepared from unknown analytes.

However, before further description of the present invention, and in order that the invention may be more readily understood, certain terms have been first defined and collected here for convenience.

### I. Definitions

The language “accuracy” is art-recognized and describes the degree of conformity of a measure, i.e., the quantity, to a standard or a true value. For example, an increase in the accuracy of analyte quantification refers to an improvement in obtaining a measured value that is closer to the actual or true value. This improvement may be identified/described by reference to a percent increase in accuracy with respect to the accuracy obtainable using existing methods of measurement that utilize mass spectroscopy of a plurality of analytes. In certain embodiments, the accuracy is increased by greater (or equal to) 10% compared to existing techniques, e.g., greater (or equal to) 12%, e.g., greater (or equal to) 14%, e.g., greater (or equal to) 16%, e.g., greater (or equal to) 18%, e.g., greater (or equal to) 20%, e.g., greater (or equal to) 30%, e.g., greater (or equal to) 40%.

As used herein, the term “amino acid” describes both naturally occurring and non-naturally occurring amino acids as well as amino acid analogs and mimetics.

Naturally occurring amino acids are art-recognized, and include the 20 natural (L)-amino acids utilized during natural protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like. Amino acid analogs include modified forms of naturally and non-naturally occurring amino acids. Such modifications can include, for example, substitution or replacement of chemical groups and moieties on the amino acid or by derivitization of the amino acid. Amino acid mimetics include, for example, organic structures that exhibit functionally similar properties such as charge and charge spacing characteristic of the reference amino acid. For example, an organic structure that mimics lysine (Lys or K) would have a positive charge moiety located in similar molecular space and having the same degree of mobility as the ε-amino group of the side chain of the naturally occurring Lys amino acid. Mimetics also include constrained structures so as to maintain optimal spacing and charge interactions of the amino acid or of the amino acid functional groups.

The term “analyte,” as used herein, refers to any chemical or biological compound or substance that is subject to the analysis of the invention capable of derivatization according to the methods of the invention. Analytes of the invention include, but are not limited to, small organic compounds, amino acids, peptides, polypeptides, proteins, nucleic acids, polynucleotides, biomarkers, synthetic or natural polymers, or any combination or mixture thereof. In certain embodiments, the analyte is a primary or secondary amino acid. In particular embodiments, moreover, it should be understood that the use of the term “analyte” as used throughout the specification may be interpreted in its singular or plural form. In certain embodiments, the plurality of analytes in the sample is greater than 5, e.g., greater than 10, e.g., greater than 15, e.g., greater than 20, e.g., greater than 25, e.g., greater than 30, e.g., greater than 35, e.g., greater than 45.

The language “analyte derivative,” as used herein, describes an analyte that is functionalized with another moiety in order to convert the analyte into a derivative thereof. It is the analyte derivative that is detected by the methods of the invention for use in determining the unknown quantity of an analyte in a sample, using a response factor calculation. In certain embodiments, the analyte is an amino acid, the derivative of which is an amino acid derivative. In certain embodiments, the number of analyte derivatives being measured is greater than or equal to 5, e.g., greater than or equal to 10, e.g., greater than or equal to 11, e.g., greater than or equal to 12, e.g., greater than or equal to 13, e.g., greater than or equal to 10, e.g., greater than or equal to 14, e.g., greater than or equal to 15, e.g., greater than or equal to 20, e.g., greater than or equal to 25, e.g., greater than or equal to 30.

The language “analyte derivative standards,” as used herein, describes analyte derivatives that are present in known quantities, which may be used as the reference point to calculate the quantity of the unknown analyte derivatives using a response factor calculation as described herein below. In certain embodiments, the analyte derivative standard is an amino acid derivative standard. In certain embodiments, the number of analyte derivative standards is greater than or equal to 5, e.g., greater than or equal to 10, e.g., greater than or equal to 11, e.g., greater than or equal to 12, e.g., greater than or equal to 13, e.g., greater than or equal to 10, e.g., greater than or equal to 14, e.g., greater than or equal to 15, e.g., greater than or equal to 20, e.g., greater than or equal to 25, e.g., greater than or equal to 30. In particular embodiments, the number of analyte derivative standards is equal to the number of analyte derivatives.

The term “analyzing” or “analysis” is used herein to describe the method by which the quantity of each of the individual analytes described herein is detected. Such analysis may be made using any technique that distinguishes between the analyte (or analyte derivative) and the analyte standard (or analyte derivative standard). In one embodiment of the invention, the analysis or act of analyzing includes mass spectrometry (MS).

The language “chromatographic separation” is art-recognized, and describes the process in which a chemical mixture carried by a liquid or gas is separated into components as a result of differential distribution of the solutes as they flow around or over a stationary liquid or solid phase. For example, chromatographic separations suitable for use in invention include, but are not limited to liquid chromatographic (including HPLC) methods such as normal-phase HPLC, RP-HPLC, HILIC, and size-exclusion chromatography (SEC), including gel permeation chromatography (GPC). Other suitable methods include additional HPLC methods and related liquid chromatographic techniques, including, e.g., ultra-performance liquid chromatography (HPLC), fast performance liquid chromatography (FPLC) and the like.

The term “derivatizing” is used herein to describe the act of functionalizing or reacting a chemical or biological compound or substance, e.g., an analyte, with another moiety in order to convert such compound or substance into a derivative thereof. In certain embodiments of the present invention, such derivatization occurs using a derivatizing agent that acts to functionalize an analyte and/or a standard i.e., AccQTag™, PicoTag®, or a functional derivative thereof. Functional derivatives of derivatizing agents AccQTag™ and PicoTag® include modifications of the chemical structure of the AccQTag™ and PicoTag® reagents that would not substantially affect the ability of these reagents to perform their intended function, i.e., derivatization and utility in detection according to the methods of the invention.

As used herein, the language “functional derivative,” for example as used in the expression “a functional derivative thereof,” describes any derivative of a molecule that retains its ability to perform its intended function, i.e., a derivative that is functional. For example, functional derivatives of AccQTag™ or PicoTag® are any derivatives of AccQTag™ or PicoTag® that have been prepared by derivatizing these moieties with any functional group that allows these compounds to retain their ability to perform their intended function in the methods of the invention.

As used herein, a “functional group” is any chemical group that has desirable functional properties. A desirable functional property is any property that imparts a desirable chemical characteristic to a molecule. A functional group can include a group that changes the physicochemical properties of a molecule, for example, changing the mass, charge, hydrophobicity, and the like. A particularly useful functional group is a label or tag, for example, fluorophores, chromophores, spin labels, isotope distribution tags, and the like.

The language “internal standard,” as used herein, describes a collection of one or more functionalized chemical or biological compounds or substances, e.g., one or more analytes functionalized with another moiety in order to convert such compounds or substances into a derivative thereof. Internal standards of the invention are present in known concentrations and added to the sample to form a sample mixture. The addition of the internal standard allows for the detection of and comparison between the known concentrations of one or more known analytes, with the unknown concentrations of analytes in the original sample. As such, the internal standards of the present invention provide a novel way to measure the absolute quantity of a plurality of analytes in sample using a response factor calculation.

In certain embodiments, the internal standard is previously prepared, and allows for direct addition into the sample to form the sample mixture. In an alternative embodiment, the internal standard is prepared just prior to addition to the sample, e.g., using automation, by reaction of a known concentration of one or more analytes with reagents capable of producing one or more (e.g., a plurality of) analyte derivative standards, including known concentrations of derivatizing agents and one or more analytes under investigation. In yet another alternative embodiment, the internal standard is prepared in situ with the sample to form the sample mixture by direct addition of reagents capable of producing, for example, a plurality of analyte derivative standards, including known concentrations of derivatizing agents and activated analytes (which would be more reactive to the derivatizing agent than analytes in the sample). The reagents used to prepare the internal standard are noted herein by the language “internal standard reagent.”

The term “isotope” is art-recognized, and describe any of the several different forms of an element each having different atomic mass. Isotopes of an element have nuclei with the same number of protons (the same atomic number) but different numbers of neutrons. “Stable isotopes” are chemical isotopes that are not radioactive. Stable isotopes of the same element have the same chemical characteristics and therefore behave almost identically. The mass differences, due to a difference in the number of neutrons, allows for a difference in detection in the methods of the invention.

As used herein, the term “isotopic label” or “isotope tag” refers to a chemical group that can be generated in two distinct isotopic forms, for example, heavy and light isotopic versions of the constituent elements making up the chemical group. Such constituent elements include, for example, carbon, oxygen, hydrogen, nitrogen, and sulfur. In addition, other elements that are chemically or functionally similar can be substituted for the above naturally occurring elements. Particularly useful isotopic labels or tags are those that allow convenient analysis by MS. For example, heavy and light isotopic versions of an amino acid can be used to differentially isotopically label a polypeptide.

As used herein, the term “label” is intended to mean any moiety that can be attached to a molecule that results in a detectable change as compared with the unlabeled molecule. The label can be bound to the molecule either covalently or non-covalently, although generally the label will be covalently bound. It is understood that, where a non-covalent interaction occurs between the label and the molecule, the non-covalent interactions are of sufficiently high affinity to allow the label to remain bound to the molecule during chemical and/or physical manipulations used in methods of the invention. The label of the present invention is detectable according to the methods of the present invention and imparts a characteristic to a molecule such that it can be detected by any of a variety of analytical methods, including MS, chromatography, fluorography, spectrophotometry, immunological techniques, and the like. A label can be, for example, an isotope, fluor, chromagen, ferromagnetic substance, luminescent label, or an epitope label recognized by an antibody or antibody fragment.

A particularly useful label is a mass label useful for analysis of a sample by MS, i.e., an isotopic label. The change in mass of the molecule due to the incorporation of a mass label should be within the sensitivity range of the instrument selected for mass determination. In addition, one skilled in the art will know or can determine the appropriate mass of a label for molecules of different sizes and different compositions. Moreover, when using heavy and light mass labels, for example, for differential labeling of molecules, a mass difference as small as between about 1-3 mass units can be used or as large as greater than about 10 mass units. Mass labels suitable for differentially labeling two samples are chemically identical but differ in mass. Exemplary mass labels include, for example, a stable isotope tag, an isotope distribution tag, a charged amino acid, differentially isotopically labeled tags, and the like. A label can also be a gas-phase basic group such as pyridyl or a hydrophobic group. A label can also be an element having a characteristic isotope distribution, for example, chlorine, bromine, or any elements having distinguishable isotopic distribution. Additionally, a label can have a bond that breaks in a collision cell or ion source of a mass spectrometer under appropriate conditions and produces a reporter ion.

The language “liquid chromatography” is art-recognized and includes chromatographic methods in which compounds are partitioned between a liquid mobile phase and a solid stationary phase. Liquid chromatographic methods are used for analysis or purification of compounds. The liquid mobile phase can have a constant composition throughout the procedure (an isocratic method), or the composition of the mobile phase can be changed during elution (e.g., a gradual change in mobile phase composition such as a gradient elution method).

The language “mass spectrometry” and “mass spectroscopy” are art-recognized and used herein, interchangeably to describe an instrumental method for identifying the chemical constitution of a substance by means of the separation of gaseous ions according to their differing mass and charge. A variety of mass spectrometry systems can be employed to analyze the analyte molecules of a sample subjected to the methods of the invention. For example, mass analyzers with high mass accuracy, high sensitivity and high resolution may be used and include, but are not limited to, atmospheric chemical ionization (APCI), chemical ionization (CI), electron impact (EI), fast atom bombardment (FAB), field desorption/field ionization (FD/FI), electrospray ionization (ESI), thermospray ionization (TSP), matrix-assisted laser desorption (MALDI), matrix-assisted laser desorption time-of-flight (MALDI-TOF) mass spectrometers, ESI-TOF mass spectrometers, and Fourier transform ion cyclotron mass analyzers (FT-ICR-MS). In addition, it should be understood that any combination of MS methods could be used in the methods described herein to analyze an analyte in a sample. In certain embodiments, the MS technique used for analysis of the analyte described herein is one that is applicable to most polar compounds, including amino acids, e.g., ESI.

The term “mobile phase” is art-recognized, and describes a liquid solvent system used to carry a compound of interest into contact with a solid phase (e.g., a solid phase in a solid phase extraction (SPE) cartridge or HPLC column) and to elute a compound of interest from the solid phase.

The language “non-volatile salts”, as used herein, describes salts present in the mobile phase which are substantially non-volatile under conditions used for removing mobile phase solvents when interfacing a liquid chromatography system with a mass spectrometer. Thus, salts such as sodium chloride or potassium phosphate are considered non-volatile salts, whereas salts such as ammonium formate, ammonium bicarbonate, or ammonium acetate, which are largely removed under vacuum, are volatile salts. Other volatile salts can be used, as will be apparent to one of ordinary skill in the art. For example, ammonium (NH₄⁺) salts of volatile acids (e.g., formic acid, acetic acid, trifluoroacetic acid, perfluorooctanoic acid) are generally volatile salts suitable for use with MS detection.

As used herein, the term “nucleic acid” describes a molecule containing two or more nucleotides covalently bonded together, such as deoxyribonucleic acid (DNA) or ribonucleic acids (RNA) and including, for example, single-stranded and a double-stranded nucleic acid. The term is similarly intended to include, for example, genomic DNA, cDNA, mRNA and synthetic oligonucleotides corresponding thereto that can represent the sense strand, the anti-sense strand or both.

The term “obtaining” as in obtaining a material, component or substance is intended to include buying, synthesizing or otherwise acquiring the material. In certain embodiments of the invention, the methods comprise an additional step of obtaining the sample or reagents for use in the methods of the invention, e.g, an AccQTag™ reagent or a PicoTag® reagent.

The term “oligonucleotide” as used herein denotes single stranded nucleotide multimers of from about 10 to 100 nucleotides and up to 200 nucleotides in length, or longer, e.g., up to about 500 nucleotides or longer. Oligonucleotides are usually synthetic and, in certain embodiments, are under 100, e.g., under 50 nucleotides in length.

As used herein, the term “polypeptide” describes a peptide of two or more amino acids. A polypeptide can also be modified by naturally occurring modifications such as post-translational modifications or synthetic modifications, including phosphorylation, lipidation, prenylation, palmitylation, myristylation, sulfation, hydroxylation, acetylation, glycosylation, ubiquitination, addition of prosthetic groups or cofactors, formation of disulfide bonds, proteolysis, assembly into macromolecular complexes, and the like.

A polypeptide includes small polypeptides having a few or several amino acids as well as large polypeptides having several hundred or more amino acids. Usually, the covalent bond between the two or more amino acid residues is an amide bond. However, the amino acids can be joined together by various other means known to those skilled in the peptide and chemical arts. Therefore, the term polypeptide is intended to include molecules that contain, in whole or in part, non-amide linkages between amino acids, amino acid analogs, and mimetics. Similarly, the term also includes cyclic polypeptides and other conformationally constrained structures.

Modified polypeptides can also include non-naturally occurring derivatives, analogues and functional mimetics thereof generated by chemical synthesis, provided that such polypeptide modification displays a similar functional activity compared to the parent polypeptide. For example, derivatives can include chemical modifications of the polypeptide such as alkylation, acylation, carbamylation, iodination, or any modification that derivatizes the polypeptide. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Moreover, free carboxyl groups can be derivatized to form salts, methyl and ethyl esters or other types of esters or hydrazides; free hydroxyl groups can be derivatized to form O-acyl or O-alkyl derivatives; and/or the imidazole nitrogen of histidine can be derivatized to form N-im-benzylhistidine. Also included as derivatives or analogues are those polypeptides which contain one or more naturally occurring amino acid derivatives of the twenty standard amino acids, for example, 4-hydroxyproline, 5-hydroxylysine, 3-methylhistidine, homoserine, ornithine or carboxyglutamate, and can include amino acids that are not linked by peptide bonds.

The term “precision” is art-recognized and describes the reproducibility of a result. It is measured by comparison of successive values obtained for a measurement to the prior values, where more precise measurements (or those with greater precision) will be demonstrated by successive measurements that are more consistently closer to the prior measurements. In certain embodiments, the precision is increased by greater (or equal to) 10% compared to existing techniques, e.g., greater (or equal to) 12%, e.g., greater (or equal to) 14%, e.g., greater (or equal to) 16%, e.g., greater (or equal to) 18%, e.g., greater (or equal to) 20%, e.g., greater (or equal to) 30%, e.g., greater (or equal to) 40%.

The term “pump” as used herein, with respect to the chromatography art, is art-recognized.

The terms “quantitative” and “quantitatively” are art-recognized and used herein to describe measurements of quantity or amount. For example, the term “quantification” describes the act of measuring the quantity or amount of a particular object, e.g., an analyte. However, in the embodiments of the present invention, the quantitative analysis is a measurement of an absolute amount, as opposed to relative amount, i.e., the total amount of analyte may be quantified absolutely in order to determine the actual amount of the analyte.

The language “response factor calculation” as used herein, describes the calculation performed to determine the quantity of an analyte in a sample utilizing the response values obtained from the detection of the analyte derivative (AD) and analyte derivative standard (ADS). The quantity of each analyte derivative is determined by multiplying the detection ratio by the ratio of response of the analyte derivative peak to the analyte derivative standard peak, by the concentration of the analyte derivative standard. This calculation results in the determination of the concentration of an analyte. Moreover, to obtain this concentration may be used to provide the amount of analyte in the sample, based on known volumes of sample tested, and/or total volume sample from which the testing sample was taken. In certain embodiments, the response factor calculation may be determined using software adapted for this purpose.

It should be noted that the detection ratio is the ratio based in accordance with the difference in detection of the derivatizing agents used to prepare the analyte derivative and analyte derivative standard, i.e., due to differences in the derivatizing agent used to create the analyte derivative as compared with the analyte derivative standard it may be expected that the ADS is detected to a different extent than the AD (wherein the detection ratio accounts for this difference). In certain embodiments, the detection ratio is 1, or alternatively stated the detection ratio of AD to ADS is a 1 to 1 ratio.

The term “sample” is used herein to describe a representative portion of a larger whole or group of components that are capable of being separated and detected by the methods of the present invention. Exemplary samples include chemically or biologically derived substances, e.g., analytes of the present invention. In particular embodiments, the components of the sample include, but are not limited to small organic compounds, amino acids, peptides, polypeptides, proteins, nucleic acids, polynucleotides, biomarkers, synthetic or natural polymers, or any combination or mixture thereof.

The language “sample mixture,” as used herein, describes the resultant product when a sample is mixed or combined with one or more analyte derivative standards, e.g., of a known concentration.

### II. Methods of the Invention

The present invention describes novel methods and systems for analyzing a compound/analyte or a mixture of compounds/analytes. The methods and systems of the invention are capable of separating and thereby resolving complex mixtures of analytes, allowing for the simultaneous identification and quantitative analysis of a plurality of components of such mixtures.

In one embodiment, the invention is directed to a method for quantitatively analyzing a plurality of analytes in a sample. The method comprises derivatizing analytes in a sample with a first derivatizing agent to form analyte derivatives in the sample, adding a known concentration of a plurality of analyte derivative standards to the sample to form a sample mixture, subjecting the sample mixture to chromatographic separation, detecting each individual analyte derivative and analyte derivative standard, and determining the quantity of each analyte derivative in the sample.

The amount of each analyte derivative is determined by a response factor calculation (RFC), in order to quantitatively analyze the plurality of analytes in the sample. In certain embodiments, one or more of the analyte derivative standards has been formed by derivatizing an analyte standard with a second derivatizing agent. In certain additional embodiments, the method further comprises the step of derivatizing a known concentration of analyte standards with a second derivatizing agent to form a known concentration of a plurality of analyte derivative standards. In particular embodiments, the detection of the analyte derivative and the corresponding analyte derivative standard is measured as a 1:1 response ratio, i.e., simplifying the RFC. Alternatively, the response ratio may not be a 1:1 ratio, for example, due to the use of unrelated derivatizing agents to produce the analyte derivative and the analyte derivative standard.

In certain embodiments, the sample to be analyzed or purified contains between about 1 and about 5000 analytes. In certain embodiments, the sample contains at least 5 analytes. In certain embodiments, the sample contains at least 10 analytes. In certain embodiments, the sample contains at least 15 analytes. In certain embodiments, the sample contains at least 20 analytes. In certain embodiments, the sample contains at least 25 analytes. In certain embodiments, the sample contains at least 30 analytes. In certain embodiments, the number of analytes (or analyte derivatives) is equal to the number of analyte derivative standards.

Such analytes may be found in complex mixtures in biological samples, which may be derived, for example, from a biological specimen (wherein the term “specimen” refers specifically to a sample obtained from an organism or individual). These specimen may be obtained from an individual as a fluid or tissue specimen. For example, a tissue specimen may be obtained from a biopsy, such as a skin biopsy, tissue biopsy or tumor biopsy. A fluid specimen may be blood, serum, urine, saliva, cerebrospinal fluid or other bodily fluids. In certain embodiments, a fluid specimen is particularly useful in methods of the invention (in that fluid specimens are readily obtained from an individual). Methods for collection of specimens are well known to those skilled in the art (see, for example, Young and Bermes, in Tietz Textbook of Clinical Chemistry, 3rd ed., Burtis and Ashwood, eds., W. B. Saunders, Philadelphia, Chapter 2, pp. 42 72 (1999)). In certain embodiments, specimen may also be a microbiological specimen, which may be derived from a culture of the microorganisms, including those cultured from a specimen from an individual.

Moreover, the analytes or compounds present in the mixture may include, for example, small organic molecules (such as pharmaceuticals or pharmaceutical candidates, typically having a molecular weight of less than 1000), amino acids, proteins, peptides or polypeptides (e.g., from peptide synthesis or from biological samples, including digests of proteins or mixtures of proteins), nucleic acids or polynucleotides (e.g., from biological samples or from synthesized polynucleotides), biomarkers, synthetic or natural polymers, or mixtures of these materials. The types of compounds are limited only by the chromatographic methods selected for compound separation, as described herein. In certain preferred embodiments, the analyte contains a primary or secondary amine, or is otherwise derivatized with a primary or secondary amine prior to subjection to the methods of the present invention.

In certain embodiments, at least one analyte is at least partially charged at a pH in the range of about 2 to about 12. More preferably, at least one compound or impurity has a first charge state at a first pH in the range of about 2 to about 12 and a second charge state at a second pH in the range of about 2 to about 12. For example, a compound could have a charge of +1 at a lower pH, and have a charge of 0 (neutral) at a higher pH; or a charge of +2 at a lower pH, a charge of +1 at a higher pH, and a charge of 0 at a third, still higher pH. In certain preferred embodiments, an analyte to be detected, analyzed, or purified is an amino acid, peptide, polypeptide, or protein.

In particular embodiments, the analytes are selected from the group consisting of amino acids, polypeptides, and mixture thereof. In another particular embodiment, the analytes are selected from the group consisting of amino acids and mixtures thereof, e.g., a primary or secondary amino acid. This amino acid may be, for example, selected from the group consisting of known natural and non-natural amino acids.

In accordance with the methods of the invention, the analytes of the invention are derivatized to form analyte derivatives, e.g., derivatives of the small organic molecule, amino acid, peptide, polypeptide, protein, nucleic acid, polynucleotide, biomarker, or polymer derivatives. These derivatives are produced by modification of the analytes to incorporate desirable functional characteristics, for example, using the methods disclosed herein. Such modifications may include the incorporation of a label or tag, e.g., labels or tags that include isotopic substitution useful for MS analysis.

An exemplary embodiment of the invention provides amino acid analytes and their respective amino acid analyte derivatives. Methods and chemistries for modifying amino acid side chains in peptides, polypeptides, or proteins are well known to those skilled in the art (see, for example, Glazer et al., Laboratory Techniques in Biochemistry and Molecular Biology: Chemical Modification of Proteins, Chapter 3, pp. 68 120, Elsevier Biomedical Press, New York (1975), which is incorporated herein by reference; and Pierce Catalog (1994), Pierce, Rockford Ill.). Any of a variety of reactive groups can be incorporated into a chemical group for reacting with a sample molecule so long as the reactive group can be covalently coupled to a molecule, for example, a polypeptide. Accordingly, a reactive group can react with carboxyl groups found in Asp or Glu; with other amino acids such as His, Tyr, Arg, and Met; with amines such as Lys, for example, imidoesters and N-hydroxysuccinimidyl esters; with oxygen or sulfur using chemistry well known in the art; with a phosphate group for selective labeling of phosphopeptides; or with other covalently modified peptides, including glycopeptides, lipopeptides, or any of the covalent polypeptide modifications disclosed herein. Additionally, one of ordinary skill in the art will know or can readily determine conditions for modifying polypeptides using known reagents, incubation conditions and time of incubation to obtain conditions optimal for modification of polypeptides or other molecules for use in methods of the invention, and as such are considered within the scope of the present invention.

Any method for modifying the amino-terminus of a polypeptide may also be used. In addition to the method exemplified herein for modifying an amino group, including the N-terminus, other methods for modifying the N-terminus are well known to those skilled in the art (see, for example, Brancia et al., Electrophoresis 22:552 559 (2001); Hoving et al., Anal. Chem. 72:1006 1014 (2000); Munchbach et al., Anal. Chem. 72:4047 4057 (2000), each of which is incorporated herein by reference).

Furthermore, the present invention allows for great structural flexibility in the choice of the derivatizing agent or label. The structure of the derivatizing agent can be deliberately selected to achieve specific objectives. For example, very polar peptides can be made more hydrophobic and therefore better retained on reverse-phase columns by the transfer of a hydrophobic tag, a strong gas-phase basic group such as pyridyl can be transferred to direct fragmentation in the collision cell of a mass spectrometer, or elements with characteristic isotope distribution such as chlorine or bromine can be added to provide distinct isotopic signatures for the labeled analytes, e.g., tagged peptides.

The methods of the invention can be readily applied to polypeptides having many different forms of post-translational modifications such as phosphorylation, glycosylation, ubiquitination, acetylation, palmitylation, myristylation, and the like. The methods of the invention can thus be used to selectively isolate other post-translationally modified molecules, including polypeptides, with concomitant transfer of various functional groups to the peptides. Selective isolation of a particular type of post-translational modification can also be achieved using methods of the invention.

Although the methods of the invention have generally been exemplified herein with amino acids, proteins, peptides and polypeptides, it is understood that any of a variety of molecules in a sample can be readily derivatized/labeled by the methods disclosed herein. In general, many classes of biomolecules such as oligonucleotides, metabolite, and the like, can be functionalized by and/or useful in the methods disclosed herein to incorporate desirable functional groups for improved qualitative or quantitative analysis. Certain embodiments of the invention provide derivatizing agents that are useful in the detection methods utilized for in-line monitoring of chromatographic separations, e.g., ultraviolet detection, and/or mass spectrometry detection. In further embodiments, such derivatizing agents are useful in derivatizing amine functional groups, e.g., primary and secondary amines.

Accordingly, in certain embodiments, an AccQTag™ reagent (Waters Corporation, Milford, Mass.) is added to a sample comprising the analytes described herein, e.g., amino acids, in order to produce analyte derivatives. Such reagents include AccQFluor™ (Waters Corporation, Milford, Mass.) which is 6-aminoquinolyl-N-hydroxysuccinimidyl carbamate, or AQC; an N-hydroxysuccinimide-activated heterocyclic carbamate. The structure of AccQFluor™ is provided below.

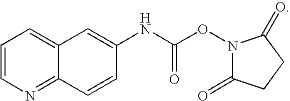

In other embodiments, a PicoTag® reagent (Waters Corporation, Milford, Mass.) is added to sample comprising amino acids. Precolumn derivatization relies on the coupling reaction of the Edman Degradation, the reaction of phenylisothiocyanate (PITC), shown below,

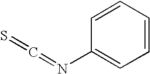

with both primary and secondary amino acids to form phenylthiocarbamyl (PTC) derivatives.

The second derivatizing agent useful in producing the analyte derivative standards of the invention may comprise an isotope. In certain embodiments, the isotope is a radioactive isotope. In other embodiments, the isotope is a stable isotope, e.g., selected from the group consisting of ¹³C, ¹⁵N, and ²H. As such, in certain embodiments, functional groups useful for derivatizing the analytes of the invention include, for example, stable isotope tags that enable accurate peptide quantification by mass spectrometry based on isotope dilution theory, isotope distribution tags that identify the tagged peptides or fragments thereof by their isotope distribution, charged amino acids, or other compounds that mediate efficient ionization in a mass spectrometer and direct the fragmentation pattern in the collision cell of a tandem mass spectrometer. In particular embodiments the second derivatizing agent is AccQTag™, PicoTag®, or a functional derivative thereof, which has been labeled with an isotope. In certain embodiments, the isotope is a radioactive isotope. In other embodiments, the isotope is a stable isotope, e.g., selected from the group consisting of ¹³C, ¹⁵N, and ²H.

In a specific embodiment, the first derivatizing agent and the second derivatizing agent are different isotopes of the same molecule, e.g., different isotopes of AccQTag™, PicoTag®, or a functional derivative thereof. Moreover, the skilled artisan would understand that different isotopes of the same molecule typically (but not always) produce a response ratio useful in the RFC that is 1:1.

Moreover, novel isotopically labeled reagents of AccQTag™ and derivatives made therewith are contemplated by the instant invention. Similarly, novel isotopically labeled reagents of PicoTag® and derivatives made therewith are contemplated by the instant invention. In one embodiment, the invention provides for isotopically labeled amino acid derivatives to be used as a standard for quantitative analysis of a sample, e.g., derivatives prepared using AccQTag™, PicoTag®, or a functional derivative thereof.

An additional aspect of the invention is an internal standard reagent useful for quantitatively analyzing an analyte comprising AccQTag™ or a functional derivative thereof, which has been labeled with an isotope, e.g., a radioactive or stable isotope.

In yet another aspect, the invention provides an internal standard useful for quantitatively analyzing an analyte prepared by reacting a known concentration of one or more analytes with AccQTag™ or a functional derivative thereof, which has been labeled with an isotope, e.g., a radioactive or stable isotope.

In another embodiment, the invention is a method of increasing precision of analyte quantification of a plurality of analytes. The method comprises derivatizing analytes in a sample with a first derivatizing agent to form analyte derivatives in the sample, adding a known concentration of a plurality of analyte derivative standards to the sample to form a sample mixture, subjecting the sample mixture to chromatographic separation, detecting each individual analyte derivative and analyte derivative standard, and determining the quantity of each analyte derivative in the sample. The amount of each analyte derivative is determined by a response factor calculation. As such, this method is useful for increasing the precision of analyte quantification of a plurality of analytes in the sample with respect to known methods. Increases in the precision may be at least 5%, e.g., at least 6%, e.g., at least 7%, e.g., at least 8%, e.g., at least 9%, e.g., at least 10%, e.g., at least 11%, e.g., at least 12%, e.g., at least 13%, e.g., at least 14%, e.g., at least 15%, compared to existing techniques.

A further embodiment of the invention is a method of increasing accuracy of analyte quantification of a plurality of analytes. The method comprises derivatizing analytes in a sample with a first derivatizing agent to form analyte derivatives in the sample, adding a known concentration of a plurality of analyte derivative standards to the sample to form a sample mixture, subjecting the sample mixture to chromatographic separation, detecting each individual analyte derivative and analyte derivative standard, and determining the quantity of each analyte derivative in the sample. The amount of each analyte derivative is determined by a response factor calculation. As such, this method is useful for increasing the accuracy of analyte quantification of a plurality of analytes in the sample with respect to known methods. Increases in the accuracy may be at least 5%, e.g., at least 6%, e.g., at least 7%, e.g., at least 8%, e.g., at least 9%, e.g., at least 10%, e.g., at least 11%, e.g., at least 12%, e.g., at least 13%, e.g., at least 14%, e.g., at least 15%, compared to existing techniques.

Furthermore, although the methods of the invention are advantageous in that complex biological samples can be analyzed directly, a sample can also be processed, if desired. For example, a blood sample can be fractionated to isolate particular cell types, for example, red blood cells, white blood cells, and the like. A serum sample can be fractionated to isolate particular types of proteins, for example, based on structural or functional properties such as serum proteins modified by glycosylation, phosphorylation, or other post-translational modifications, or proteins having a particular affinity, such as an affinity for nucleic acids. A serum sample can also be fractionated based on physical-chemical properties, for example, size, pI, and the like. A serum sample can additionally be fractionated to remove bulk proteins present in large quantities, such as albumin, to facilitate analysis of less abundant serum polypeptides. Furthermore, a cellular sample can be fractionated to isolate subcellular organelles. Moreover, a cellular or tissue sample can be solubilized and fractionated by any of the well known fractionation methods, including chromatographic techniques such as ion exchange, hydrophobic and reverse phase, size exclusion, affinity, hydrophobic charge-induction chromatography, and the like (Ausubel et al., supra, 1999; Scopes, Protein Purification: Principles and Practice, third edition, Springer-Verlag, New York (1993); Burton and Harding, J. Chromatogr. A 814:71 81 (1998)).

The methods of the invention are particularly useful for identification and quantitative analysis of the molecules contained in biological samples, and in particular for the analysis of amino acids. Such analysis includes derivatizing a sample, adding a known amount of an internal standard, and detecting and determining the quantity of each analyte in the sample. The invention also provides reagents that are useful for internal standards for LC/MS analysis.

### II. Chromatography and Systems for Use Therewith

The present invention may utilize any suitable chromatographic methods and systems, e.g., known or introduced/refined herein. For example, in certain embodiments the chromatographic separation is liquid chromatography, e.g., high performance liquid chromatography (HPLC).

Typical systems of the invention comprise a chromatographic column, an analysis system, and agents/reagents useful for the analysis of multiple analytes simultaneously. For example, in one embodiment, the invention is directed to a liquid chromatography/mass spectroscopy system for quantitatively analyzing the amount of a plurality of analytes in a sample. The system contains a chromatographic analysis system comprising a chromatographic column and a pump for pumping at least one mobile phase through the chromatographic column.

The system also contains a mass spectroscopy analysis system comprising a mass spectrometer capable of detecting analyte derivatives, a first derivatizing agent useful for derivatizing analytes in a sample to form analyte derivatives in the sample comprising AccQTag™ or a functional derivative thereof, and a plurality of analyte derivative standards comprising AccQTag™ or a functional derivative thereof that have been labeled with an isotope. In certain embodiments, the isotope is a radioactive isotope. In other embodiments, the isotope is a stable isotope, e.g., selected from the group consisting of ¹³C, ¹⁵N, and ²H. In certain embodiments, the number of analyte derivative standards is greater than 5, e.g., greater than 10, e.g., greater than 15, e.g., greater than 20. In certain embodiments, the analyte derivative standard is between 5 and 40, e.g., between 5 and 30, e.g., between 10 and 30, e.g., between 10 and 25, e.g., between 10 and 20.

Another aspect of the invention is directed to a liquid chromatography/mass spectroscopy system for quantitatively analyzing the amount of a plurality of analytes in a sample. The system contains a chromatographic analysis system comprising a chromatographic column and a pump for pumping at least one mobile phase through the chromatographic column. The system also contains a mass spectroscopy analysis system comprising a mass spectrometer capable of detecting analyte derivatives, a first derivatizing agent useful for derivatizing analytes in a sample to form analyte derivatives in the sample comprising AccQTag™ or a functional derivative thereof, and reagents capable of producing a plurality of analyte derivative standards comprising AccQTag™ or a functional derivative thereof that have been labeled with an isotope, e.g., a radioactive or stable isotope. In certain embodiments, the number of analyte derivative standards is greater than 5, e.g., greater than 10, e.g., greater than 15, e.g., greater than 20. In certain embodiments, the analyte derivative standard is between 5 and 40, e.g., between 5 and 30, e.g., between 10 and 30, e.g., between 10 and 25, e.g., between 10 and 20.

Columns suitable for performing separations according to the methods and systems of the invention are known in the art and can be selected without undue experimentation. For example, RP-HPLC columns include C₈, C₁₈, and phenyl-substituted solid supports. Normal-phase columns can employ silica as the stationary phase. HILIC separations are generally performed using a silica-based column material, optionally modified with, e.g., aminopropyl or diol modifiers. Pre-packed or coated columns or capillaries are available from commercial sources; selection of a particular stationary phase or solid support for use in a separation can be made according to factors such as the amount and complexity of the mixture to separated, the type of analyte to be determined, and the like.

Similarly, the size of the column can be selected according to factors such as the amount of sample to be analyzed or purified. For analysis of larger sample quantities, an HPLC column having a diameter of about 3 mm to about 20 mm may be used. For very small amounts of sample, a microbore column, capillary column, or nanocolumn may be used. As such, in certain embodiments, the chromatographic separation is performed using a column selected from the group consisting of a microbore column, a capillary column, a preparative column, or a nanocolumn.

Reversed phase chromatography utilizes a non-polar stationary phase in conjunction with more polar, largely aqueous mobile phases. Because sample retention in this case is driven by hydrophobic interaction, a strong mobile phase, i.e., one which can easily elute the sample from the stationary phase, will be one having a high percentage of organic solvent. Conversely, a weak mobile phase will have a lower percentage of organic solvent in reversed phase chromatography.

Normal-phase chromatography utilizes a stationary phase that is more polar than the mobile phase. A common application of normal-phase chromatography is seen in the use of a polar stationary phase, such as silica or alumina, with a mobile phase having a high percentage of organic solvent. In normal-phase, a weak mobile phase would have a high percentage of organic solvent, while a strong mobile phase would have a lower percentage of organic solvent.

In ion-exchange chromatography, retention of the sample on the stationary phase is controlled through the interaction of charged analytes with oppositely charged functional groups on the stationary phase surface. Because both the sample components and the stationary phase could contain either cation or anion exchange groups (and possibly both) these separations are strongly influenced by changes in mobile phase pH and/or ionic strength. In the case of ion-exchange separations, raising or lowering the pH and/or ionic strength of the mobile phase results in either an increase or a decrease in the elution strength of the mobile phase, depending on the pKa of the sample and whether the stationary phase is a cation or anion exchanger. The pH and/or the ionic strength may be raised or lowered as the separation requires, thereby adjusting the elution strength of the mobile phase. A large application area is the separation of biopolymers, specifically proteins and peptides.

Depending on the retention mechanism being used, many different mobile phase properties can be used to adjust mobile phase strength. In particular, the solvents may be selected so as to adjust the strength of the mobile phase. The types of solvents used are well known to those skilled in the art. For example, in both “reversed phase” and “normal phase” chromatography, the ratio of organic solvent to water in the mobile phase is typically modified to adjust the strength of the mobile phase. However, in the case of an ion-exchange-based separation, mobile phase pH and/or ionic strength is commonly manipulated to adjust the strength of the mobile phase.

In certain embodiments, one or more mobile phases are utilized. In a further embodiment, two mobile phases are utilized by the chromatographic method. Alternatively, the mobile phase comprises a mixture of two solvents, e.g., wherein the ratio of a first solvent is from about 5% to about 95%. In certain instances, the difference between the pH of the mobile phase and the pH and the second mobile phase is at least 3 pH units; e.g., if the pH of the first mode mobile phase is 2.5, then the pH of the second mode mobile phase can be at least 5.5. In certain embodiments, the pH difference is at least about 4 pH units, 5 pH units or 6 pH units. In particular embodiments, the mobile phase is selected from the following solvents: water, methanol, ethanol, isopropanol, acetonitrile, ethyl acetate, methylene chloride, diethyl ether, methyl t-butyl ether, benzene, toluene, pentane, hexane, heptane, and mixtures thereof.

In certain embodiments, the separation mode is one in which the mobile phase is compatible with analytical techniques such as mass spectrometry, e.g., the mobile phase is suitable for injection into a mass spectrometer with little or no sample clean-up or desalting. Therefore, in certain embodiments, the mobile phase is substantially free of non-volatile salts; for example, in certain embodiments, the mobile phase comprises less than about 20 mM (or less than 10 mM or 5 mM) of non-volatile salts. Thus, salts such as sodium chloride or potassium phosphate are considered non-volatile salts, whereas salts such as ammonium formate, ammonium bicarbonate, or ammonium acetate, which are largely removed under vacuum, are volatile salts. Other volatile salts can be used, as will be apparent to one of ordinary skill in the art. For example, ammonium (NH₄⁺) salts of volatile acids (e.g., formic acid, acetic acid, trifluoroacetic acid, perfluorooctanoic acid) are generally volatile salts suitable for use with MS detection.

Additionally, the detection of each analyte derivative and analyte derivative standard may be performed using mass spectrometry. As such, a chromatographic analysis system of the present invention may comprise a mass spectroscopy analysis system. In this regard, the mass spectrometry may be selected from the group consisting of atmospheric pressure chemical ionization (APCI), chemical ionization (CI), electron impact (EI), fast atom bombardment (FAB), field desorption/field ionization (FD/FI), thermospray ionization (TSP), matrix-assisted laser desorption/ionization mass spectroscopy (MALDI), matrix-assisted laser desorption/ionization time of flight mass spectroscopy (MALDI-TOF), and electrospray ionization (ESI). In particular embodiments, the mass spectrometry is electrospray ionization (ESI).

In one embodiment, the LCMS system of the invention is preferably operated through a computerized control and data analysis system, e.g., configured with software effective for operating the hardware of the chromatography (sampling systems, injection valves, mobile-phase pumps, detection systems), and for effecting tracking and acquiring data from the hardware. Suitable software is commercially available, for example, from liquid chromatography systems manufacturers, such as Waters (Milford Mass.), and/or from software manufacturers, such as Lab View brand software. The software can additionally include control elements for operating robotic fluid handlers and other devices that may be integrated into the LCMS system.

Moreover, the methods of the invention can be readily adapted to automation. For example, automated sampling, robotics, or any suitable automation methods can be applied to methods of the invention, if desired. Since all the reactions can be done easily in an automated fashion, the methods of the invention would allow for a high throughput sample preparation.

In addition, since there is virtually no sample handling such as transferring steps, loss of captured molecules is minimized, thus improving the yield of molecule recovery. The captured molecules can also be extensively washed to remove non-captured sample molecules or any regents since the captured sample molecules remain bound to the solid support during the wash steps. The methods of the invention can be used to capture essentially all of a class or multiple classes of molecules from a sample, or a portion of the molecules from a sample, as desired.

### III. Internal Standards

The present invention also includes both internal standards and internal standard reagents useful for quantitatively analyzing an analyte. For example, in one embodiment, the invention is an internal standard reagent useful for quantitatively analyzing an analyte comprising AccQTag™ or a functional derivative thereof, which has been labeled with an isotope, e.g., a radioactive or stable isotope.

In yet another embodiment, the invention provides an internal standard useful for quantitatively analyzing an analyte prepared by reacting a known concentration of one or more analytes with AccQTag™ or a functional derivative thereof, which has been labeled with an isotope, e.g., a radioactive or stable isotope.

Another embodiment provides a packaged kit comprising an internal standard and/or an internal standard reagent described herein as useful for quantitatively analyzing an analyte. The kit may also comprise instructions for use in performing simultaneous quantitative analysis on multiple analytes.

### EXEMPLIFICATION

The following example(s) are demonstrative of the utility of the internal standards of the present invention for use in absolute quantitative LC/MS analysis. These examples are for the purpose of illustration only and are not to be construed as limiting the scope of the invention in any manner.

### Example 1

### Absolute Quantitative Analysis of Multiple Analytes

In cases where the quantity of multiple analytes is to be determined (such as amino acids for clinical diagnosis where as many as 45 amino acids are of interest) it becomes expensive and cumbersome to obtain isotopically labeled standards for all the analytes. To get around this issue, existing technique require the use of only a subset of isotopically labeled standards and quantitate multiple analytes against one standard. This compromise can result in poor accuracy and precision for analytes that are quantitated against an isotopically labeled standard of a different analyte.

However, based on the teachings herein, it is possible to use an isotopically labeled version of a derivatizing reagent to derivatize a standard of the unlabelled analytes. The standard analytes will then exist as the isotopically labeled form. A known amount of the standard analytes derivatized with the labeled form of the derivatizing reagent can be added to an unknown sample that was derivatized with the unlabelled version of the derivatizing reagent. Quantitation is then accomplished as described hereinabove using the response factor calculation.

The analysis of a clinical sample (containing multiple analytes) to be analyzed begins by derivatization of the analytes in the sample with an unlabelled version of the AccQFluor reagent. A standard of known concentration of the unlabelled amino acids is also derivatized with an isotopically labeled version of the AccQFluor reagent. Furthermore, a known amount of the isotope-labeled derivatized standard is subsequently added to the derivatized sample, and quantitation is achieved by multiplying the ratio of the response of the sample to the response of the standard and multiplying by the standard concentration.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

In addition, novel equivalents to number values provided herein are intended to include number values that are one or two integers removed from the number provided herein, e.g., wherein the number of analytes in the sample is greater than 20 is also intended to include 18, 19, 21, and 22.

Moreover, although a number of embodiments of this invention have been described herein, it is apparent that the basic constructions can be altered to provide other embodiments that utilize the processes and compositions of this invention. Therefore, it will be appreciated that the scope of this invention is to be defined by the claims appended hereto and the specification as whole, rather than by the specific embodiments that have been presented hereinbefore by way of example.

1. A method for quantitatively analyzing a plurality of analytes in a sample comprising:
a) derivatizing analytes in a sample with a first derivatizing agent to form analyte derivatives in the sample;
b) adding a known concentration of a plurality of analyte derivative standards to the sample to form a sample mixture;
c) subjecting the sample mixture to chromatographic separation;
d) detecting each individual analyte derivative and analyte derivative standard; and
e) determining the quantity of each analyte derivative in the sample, wherein the amount of each analyte derivative is determined by a response factor calculation,
such that the plurality of analytes in the sample is quantitatively analyzed.

2. The method of claim 1, wherein the analyte derivative standard has been formed by derivatizing an analyte standard with a second derivatizing agent.

3. The method of claim 1, further comprising the step of derivatizing a known concentration of analyte standards with a second derivatizing agent to form the known concentration of a plurality of analyte derivative standards.

4. The method of claim 1, wherein the detection of the analyte derivative and the corresponding analyte derivative standard is measured as a 1:1 response ratio.

5. The method of claim 1, wherein the first derivatizing agent is AccQTag™, PicoTag®, or a functional derivative thereof and the second derivatizing agent is AccQTag™, PicoTag®, or a functional derivative thereof, which has been labeled with an isotope.

6. The method of claim 2, wherein the second derivatizing agent comprises an isotope.

7. The method of claim 6, wherein the isotope is a radioactive isotope or a stable isotope.

8. The method of claim 7, wherein the isotope is a stable isotope selected from the group consisting of ¹³C, ¹⁵N, and ²H.

9. (canceled)

10. (canceled)

11. The method of claim 5, wherein the isotope is a stable isotope selected from the group consisting of ¹³C, ¹⁵N, and ²H.

12. The method of claim 4, wherein the first derivatizing agent and the second derivatizing agent are different isotopes of the same molecule.

13. The method of claim 4, wherein the quantity of each analyte derivative is determined by multiplying the ratio of response of the analyte derivative peak to the analyte derivative standard peak, by the concentration of the analyte derivative standard.

14. The method of claim 1, wherein the number of analyte derivatives is equal to the number of analyte derivative standards.

15. The method of claim 1, wherein the number of analytes in the sample is greater than 5, greater than 10, greater than 15, greater than 20, greater than 25, greater than 30, greater than 35, greater than 40 or greater than 45.

16-22. (canceled)

23. The method of claim 1, wherein each sample analyte is a primary or secondary amino acid.

24. The method of claim 23, wherein the plurality of analytes in a sample is selected from the group consisting of small organic molecules, amino acids, peptides, polypeptides, proteins, nucleic acids, polynucleotides, biomarkers, synthetic or natural polymers, and mixtures thereof.

25. The method of claim 2, wherein the analytes is selected from the group consisting of amino acids and mixture thereof.

26. The method of claim 3, wherein the amino acid is selected from the group consisting of known natural and non-natural amino acids.

27. The method of claim 1, wherein the detection of each analyte derivative and analyte derivative standard is performed using mass spectrometry.

28. The method of claim 27 wherein the mass spectrometry is selected from the group consisting of atmospheric pressure chemical ionization (APCI), chemical ionization (CI), electron impact (EI), fast atom bombardment (FAB), field desorption/field ionization (FD/FI), thermospray ionization (TSP), matrix-assisted laser desorption/ionization mass spectroscopy (MALDI), matrix-assisted laser desorption/ionization time of flight mass spectroscopy (MALDI-TOF), and electrospray ionization (ESI).

29-33. (canceled)

34. The method of claim 1, wherein the chromatographic separation is performed using a microbore column, a capillary column, a preparative column, or a nanocolumn.

35. The method of claim 1, wherein the chromatographic separation comprises at least one mobile phase selected from the following solvents: water, methanol, ethanol, isopropanol, acetonitrile, ethyl acetate, methylene chloride, diethyl ether, methyl t-butyl ether, benzene, toluene, pentane, hexane, heptane, and mixtures thereof.

36. (canceled)

37. The method of claim 35, wherein the mobile phase comprises a mixture of two solvents, wherein the ratio of the first solvent is from about 5% to about 95%.

38. (canceled)

39. The method of claim 35, wherein the at least one mobile phase is substantially free of non-volatile salts.

40. (canceled)

41. The method of claim 1, wherein the plurality of analytes comprises a plurality of amino acids.

42. A liquid chromatography/mass spectroscopy system for quantitatively analyzing the amount of a plurality of analytes in a sample comprising:
a) a chromatographic analysis system comprising a chromatographic column and a pump for pumping at least one mobile phase through the chromatographic column;
b) a mass spectroscopy analysis system comprising a mass spectrometer capable of detecting analyte derivatives;
d) a first derivatizing agent useful for derivatizing analytes in a sample to form analyte derivatives in the sample comprising AccQTag™ or a functional derivative thereof; and
e) (i) a plurality of analyte derivative standards comprising AccQTag™ or a functional derivative thereof that have been labeled with an isotope; or
(ii) reagents capable of producing the plurality of analyte derivative standards comprising AccQTag™ or a functional derivative thereof that have been labeled with an isotope.

43-48. (canceled)

49. A method of increasing precision or accuracy of analyte quantification of a plurality of analytes comprising
a) derivatizing analytes in a sample with a first derivatizing agent to form analyte derivatives in the sample;
b) adding a known concentration of a plurality of analyte derivative standards to the sample to form a sample mixture;
c) subjecting the sample mixture to chromatographic separation;
d) detecting each individual analyte derivative and analyte derivative standard; and
e) determining the quantity of each analyte derivative in the sample, wherein the amount of each analyte derivative is determined by a response factor calculation,
such that the precision of analyte quantification of a plurality of analytes in the sample is increased.

50. (canceled)

51. (canceled)

52. An internal standard reagent useful for quantitatively analyzing an analyte comprising AccQTag™ or a functional derivative thereof, which has been labeled with an isotope.

53. (canceled)

54. (canceled)

55. An internal standard useful for quantitatively analyzing an analyte prepared by reacting a known concentration of one or more analytes with AccQTag™ or a functional derivative thereof, which has been labeled with an isotope.

56. (canceled)

57. (canceled)
